(19) United States
(12) Patent Application Publication
(10) Pub. No.: US 2015/0315681 A1
(43) Pub. Date: Nov. 5, 2015

(54) NOVEL NITINOL ALLOYS AND USES THEREOF IN SURGICAL IMPLANTS

(71) Applicants: Norman MUNROE, Miami, FL (US); Ryszard ROKICKI, Macungie, PA (US); Chandan PULLETIKURTHI, Miami, FL (US); Sharan RAMASWAMY, Miami, FL (US); Waseem HAIDER, Edinburg, TX (US); Puneet K.S. GILL, Anaheim, CA (US)

(72) Inventors: Norman MUNROE, Miami, FL (US); Ryszard ROKICKI, Macungie, PA (US); Chandan PULLETIKURTHI, Miami, FL (US); Sharan RAMASWAMY, Miami, FL (US); Waseem HAIDER, Edinburg, TX (US); Puneet K.S. GILL, Anaheim, CA (US)

(73) Assignee: University Park, PC511, Miami, FL (US)

(21) Appl. No.: 14/705,597

(22) Filed: May 6, 2015

Related U.S. Application Data
(60) Provisional application No. 61/987,848, filed on May 2, 2014.

(51) Int. Cl. C22C 30/00; A61F 2/24; C22C 19/05; C22C 14/00; C22C 19/03; A61F 2/82; A61F 2/01

(52) U.S. Cl. CPC C22C 30/00; A61F 2/82; A61F 2/24; A61F 2/01; C22C 14/00; C22C 19/03; C22C 19/058

(57) ABSTRACT

The current invention provides novel nitinol alloys, particularly, nitinol alloys containing a third metallic element referred to as ternary nitinol alloys. Accordingly, the current invention provides nitinol alloys including, but not limited to, Nickel-Titanium-Chromium (NiTiCr) and Nickel-Titanium-Tantalum (NiTiTa). The current invention also provides implants manufactured from the ternary nitinol alloys. The implants comprise the ternary nitinol alloys and are, optionally, surface treated to promote anti-thrombogenicity and biocompatibility, for example, through magnetoelectropolishing (MEP). Accordingly, the current invention provides nitinol alloys and implants comprising the nitinol alloys that reduce the risk of clotting due to stagnant blood flow, eliminate flushing, and minimize infection and damage to blood vessels.

### CROSS-REFERENCE TO RELATED APPLICATIONS

This application claims the priority benefit of U.S. Provisional Application Ser. No. 61/987,848, filed May 2, 2014, which is incorporated herein by reference in its entirety.

### GOVERNMENT SUPPORT

The subject invention was made with government support under a research project supported by The National Institutes of Health under Award Number 5SC3GM084816-04. The government has certain rights in this invention.

### BACKGROUND OF INVENTION

Metals and metal alloys such as nickel, titanium, stainless steel, and chromium-cobalt alloys are widely used in blood-contacting devices such as stents, vascular access components, needles, heart valve prostheses, catheters, and other permanent and temporary cardiovascular implants. Nitinol is a commonly used alloy of nickel and titanium where the two elements are generally present in roughly equal amounts. Worldwide over 2 million patients have stents manufactured predominantly from stainless steel (SS 316) and from nitinol alloys.

Unfortunately, cardiovascular implants such as stents and vascular access devices are associated with a number of significant deleterious health events. For example, stent thrombosis has accounted for about a 10% fatality rate. Use of antithrombotic drugs and dual antiplatelet regimes (heparin, aspirin, clopidogrel, prasugrel, etc.) have been reported to reduce and control early (24 hours to 30 days) and late (more than a year) stent thrombosis in bare metallic stents (BMS) and drug eluting stents respectively; however, prolonged (minimum 4-6 months and sometimes life-time) usage of these antithrombotic drugs can lead to major bleeding complications, renal failure, and diabetes.

Thrombosis is the primary cause of vascular access failure in dialysis patients. At least 41% of the central venous catheters (CVC), which play a major role in oncology, urology, and general medicine, result in thrombotic occlusion of blood vessels.

An ideal biomaterial for cardiovascular implants would resist the formation of thrombus and inflammatory reactions, at least until a proper endothelial layer is formed. Moreover, during and immediately following implantation, disruption of the endothelial layer can trigger the adhesion of proteins such as fibrinogen, fibronectin, vitronectin, immunoglobulin, and von Willebrand factor (vWF) (a blood glycoprotein) onto the newly exposed sub-endothelial layer, which can ultimately lead to activation, adhesion, and deposition of platelets and subsequent thrombus formation.

Implants having surfaces that contact the blood flow can initiate the activation, secretion, adherence, and aggregation of platelets and trigger subsequent plasmatic coagulation and immunological responses. These platelets and the platelet-derived secretion can spread, which leads to the formation of hematosis and further platelet aggregation. Indeed, the majority of blood contacting implants are prone to clotting and inflammatory responses, which impair their performance. Migration of thrombus to brain vasculature can lead to stroke and, in some cases, death of the patient.

Hemocompatibility of a biomaterial is mainly dependent on its surface characteristics, which dictate its interactions with blood. Surface properties such as alloy composition, roughness, wettability, surface free energy, and morphology impact the hemocompatibility of an implant material. Additionally, in the realm of metallic biomaterials, surface polishing can be an important factor affecting the properties of the biomaterial and surgical implants made from the biomaterial.

Sawyer et. al. showed that thrombosis can also be initiated by an electron transfer process between the surface of a biomaterial and fibrinogen in the blood, leading to a clotting cascade at anodic sites. In the case of cardiovascular stents, thrombogenicity is dependent on intrinsic properties such as corrosion resistance, hemocompatibility, and mechanical dexterity. However, the extrinsic properties of a stent such as its dimensions, design, combination of the drug and polymer coating, its placement relative to the vessel wall, which imposes specific flow disruptions such as stagnation and recirculation, also affect its thrombogenicity. Furthermore, corrosion of the implant may lead to the release of metal ions such as Ni, Co, and Cr, which can also trigger activation of leukocytes and subsequent inflammation.

The current invention provides new alloys that help avoid the deleterious effects associated with currently-used materials.

### BRIEF SUMMARY

The current invention provides unique and advantageous nitinol alloys, particularly, nitinol alloys containing a third metallic element. The nitinol alloys containing the third metallic elements are referred to herein as “ternary nitinol alloys.” Non-limiting examples of the third metallic element include Chromium (Cr) and Tantalum (Ta). Accordingly, in specific embodiments, the current invention provides nitinol alloys including, but not limited to, Nickel-Titanium-Chromium (NiTiCr) and Nickel-Titanium-Tantalum (NiTiTa).

The current invention further provides implants comprising the ternary nitinol alloys. In certain embodiments, the implants come in contact with the vascular blood flow of a subject receiving the implant. The implants can be, for example, cardiovascular and endovascular implants.

In one embodiment, the implants comprising a ternary nitinol alloy are surface treated to promote anti-thrombogenicity and biocompatibility. In a preferred embodiment the surface treatment is magnetoelectropolishing (MEP).

Accordingly, the current invention provides nitinol alloys and implants comprising the nitinol alloys that reduce the risk of clotting due to stagnant blood flow, eliminate flushing, and minimize infection and damage to blood vessel due to repeated access.

### BRIEF DESCRIPTION OF THE DRAWINGS

FIG. 1 shows platelets adhered on different metallic substrates after the platelet adhesion test.

FIG. 2 shows platelet adhesion on MP and MEP nitinol alloys with respect to surface chemistry (oxide), work of adhesion (W), and contact angle (CA).

FIG. 3 shows highlighted nuclei and mitochondria on NiTi10Cr alloy.

FIG. 4 shows highlighted nuclei and mitochondria on NiTi alloy.

FIG. 5 shows highlighted nuclei and mitochondria on NiTi10Ta alloy.

FIG. 6 shows XRD analysis of MEP treated NiTi10Ta alloy.

FIG. 7 shows XRD analysis of MEP treated NiTi5Cr alloy.

### DETAILED DISCLOSURE

The current invention provides novel nitinol alloys, particularly, ternary nitinol alloys. Nitinol, as used herein, refers to an alloy of nickel and titanium. Accordingly, a ternary nitinol alloy is a nitinol alloy further comprising a third metal element.

Nitinol consists of nickel and titanium and can contain about 40% nickel to about 60% nickel and about 40% titanium to about 60% titanium. Preferably, nitinol consists of nearly equal amounts of nickel and titanium. A ternary nitinol alloy comprises a third metal element, thereby reducing the percentage of the sum of titanium and nickel.

In certain embodiments of the current invention, the ternary nitinol alloy comprises about 80 atomic percent to about 99 atomic percent of nickel and titanium taken together and about 1 atomic percent to about 20 atomic percent of the third metal element. In one embodiment of the invention, the ternary nitinol alloy comprises about 1, 2, 3, 4, 5, 6, 7, 8, 9, 10, 11, 12, 13, 14, 15, 16, 17, 18, 19, or about 20 atomic percent of the third metal element.

For the purpose of this invention, the term atomic percent (at %) indicates the percentage of one kind of atom relative to the total number of atoms.

In certain other embodiments of the current invention, the ternary nitinol alloy comprises about 80 weight percent to about 99 weight percent of nickel and titanium taken together and about 1 weight percent to about 20 weight percent of the third metal element. In one embodiment of the invention, the ternary nitinol alloy comprises about 1, 2, 3, 4, 5, 6, 7, 8, 9, 10, 11, 12, 13, 14, 15, 16, 17, 18, 19, or about 20 weight percent of the third metal element.

For the purpose of this invention, the term weight percent (wt %) indicates the weight percentage of one element relative to the total weight.

In certain embodiments of the current invention, the third metal element is chromium or tantalum. Accordingly, the current invention provides NiTiCr alloys and NiTiTa alloys. Certain specific embodiments provide ternary nitinol alloys comprising about 1% to about 20%, about 5% to about 10%, or about 10% Cr. Certain other embodiments of the current invention provide ternary nitinol alloy comprising about 1% to about 20%, about 5% to about 10%, or about 5% Ta.

Ternary nitinol alloys of the current invention can be formed into various objects or can be used to coat various objects. The ternary nitinol alloys of the current invention provide desirable qualities to the objects produced therefrom.

In one embodiment, the objects comprising the ternary nitinol alloy are implants. The implants can be produced exclusively or almost exclusively using the ternary nitinol alloys of the current invention, or the implants can be produced from another material and coated with the ternary nitinol alloys of the current invention thereby providing implants comprising the ternary nitinol alloy only on their surfaces.

The implants containing the ternary nitinol, either exclusively or only on their surfaces, can be treated to impart desirable qualities to the surface. Examples of such treatments include, but are not limited to, magnetoelectropolishing (MEP) or mechanical polishing (MP). In a preferred embodiment, the implants comprising the ternary nitinol alloys of the current invention are surface treated with MEP.

The ternary nitinol alloy of the current invention can be used to manufacture implants that come in contact with the vascular blood flow of the subject receiving the implant. The implants may be, for example, cardiovascular implants or endovascular implants. Non-limiting examples of such implants include vascular access devices, needles, heart valve prostheses, catheters, arteriovenous fistula, bare metal stents, drug eluting stents, blood clot retrievers, vena cava filters, and endoscopes.

Materials currently used in the manufacture of implants are prone to corrosion, thrombous formation, and nickel ion release that can lead to necrosis. MEP ternary nitinol alloys are less thrombogenic, more corrosion resistant and less likely to release nickel ions. In-vitro thrombogenicity tests of the alloys of the current invention revealed that significantly fewer platelets adhered on MEP nitinol alloys as compared with untreated binary, ternary nitinol alloys, and stainless steel (SS 316). Additionally, superior confluent endothelial cell growth was observed on the ternary nitinol alloys as compared with that on the binary nitinol.

The addition of 10 Wt % tantalum (Ta) to nitinol increased the flexibility (79 GPa) and decreased the hardness (3 GPa) of the resultant ternary nitinol alloy. Addition of 5 Wt % chromium (Cr) to nitinol decreased the flexibility (97 GPa) and increased the hardness (6.3 GPa) of the resultant ternary nitinol alloys.

The alloy composition and surface treatment can directly modulate the surface characteristics. The surface characteristics of nitinol affect the hemocompatibility and biocompatibility of the implants made therefrom. For example, the addition of Cr, a highly passivating element to nitinol, is hereby shown to enhance hemocompatibility, corrosion resistance, and improved endothelial cell proliferation in implants made from the ternary nitinol containing Cr.

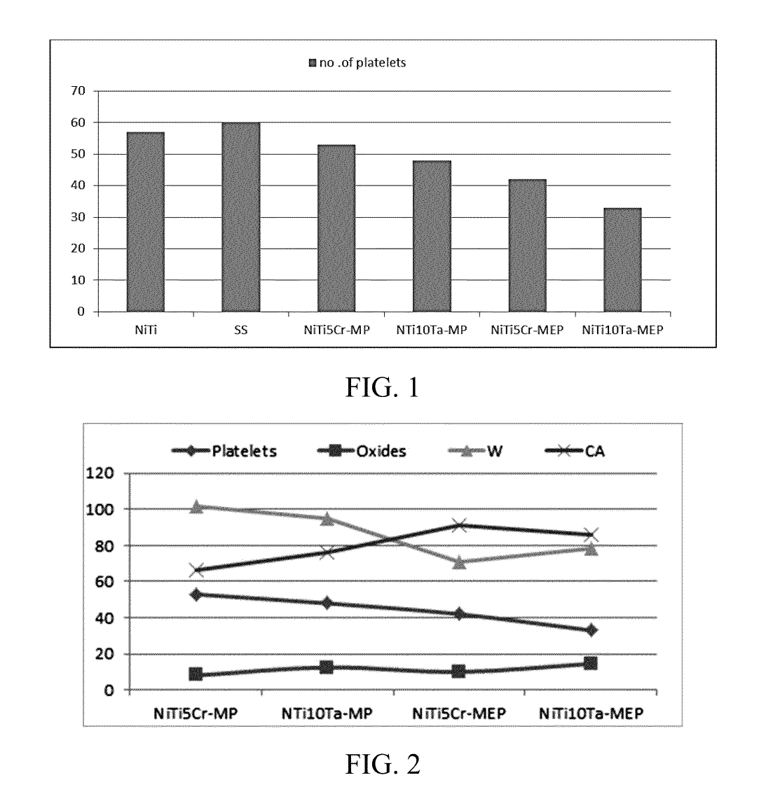

MEP processing further enhances the utility of the ternary nitinol alloys of the current invention. The hemocompatibility and biocompatibility of MEP treated ternary nitinol alloys is superior to traditional metallic biomaterial counterparts, for example, stainless steel or nitinol, that were treated with an MP process. In-vitro thrombogenicity tests revealed that significantly fewer platelets adhered on ternary nitinol alloys treated with MEP process as compared with those treated with MP process (FIG. 1).

These superior hemocompatibility and biocompatibility properties may be due to the formation of a thin, compact, and mixed hydrophobic oxide layer that formed during MEP process. As such, ternary nitinol alloys of the current invention, particularly those treated with MEP process, demonstrate enhanced suitability for use in blood-contacting applications and provide novel and superior materials for manufacturing implants, particularly, implants coming in contact with the vascular blood flow of the subjects receiving the implants.

Because of the favorable biocompatibility characteristics of the implants of the current invention it is possible to use these implants without, or with less, use of antithrombotic drugs and/or antiplatelet treatments.

The term “about” is used in this patent application to describe some quantitative aspects of the invention, for example, percentage of a metal in an alloy. It should be understood that absolute accuracy is not required with respect to those aspects for the invention to operate. When the term “about” is used to describe a quantitative aspect of the invention the relevant aspect may be varied by ±10% (e.g., ±1%, ±2%, ±3%, ±4%, ±5%, ±6%, ±7%, ±8%, ±9%, or ±10%).

### Materials and Methods

Stainless steel (SS), binary nitinol (NiTi), ternary nitinol alloys (NiTi5Cr and NiTi10Ta), and MEP treated ternary nitinol alloys were used as test specimens.

### Mechanical Polishing

Metallic samples (NiTi and SS square samples of ˜2 cm×2 cm and 0.5 mm thick); NiTi5Cr (nitinol comprising 5% Cr) and NiTi10Ta (nitinol containing 10% Ta) circular discs of about 1 cm in diameter and about 2 mm thick) of the aforementioned materials were MP treated using 3 different waterproof silicon carbide papers namely, 320 followed by 1200 and 4000 using a plate grinder at 30 rpm for 10 minutes.

### MEP Process

The MEP process utilizes an externally applied uniform magnetic field below 500mT (produced by a neodymium ring) around an electrolytic cell to achieve a smooth surface with improved and uniform corrosion resistance. The quality of MEP treatment is dependent on parameters such as duration of the process, voltage level, type of electrolyte and its temperature. MEP is conducted below the oxygen evolution regime, which not only prevents hydrogen absorption but also removes residual hydrogen from the metal. Both MP and MEP samples were ultrasonically cleaned in water, acetone, and ethyl alcohol for 5 minutes each, prior to conducting contact angle and platelet adhesion studies.

### Formation of Passive Oxide Layer

Titanium oxide (TiO₂) is the most stable of the Ti metal oxides (TiO, Ti₂O₃, Ti₃O₅) formed on the surface of nitinol and titanium alloys and is responsible for their corrosion resistance. The common crystal structures of TiO₂ are: rutile-tetragonal; anatase-tetragonal, amorphous; and brookite-orthorombic. During MEP treatment, the native oxide layer is removed from the surface of the nitinol and dissolved oxygen is adsorbed onto the metal surface. A potential drop develops across the interface and the bulk of the alloy as electrons from Ti atoms diffuse towards the adsorbed oxygen ions. This creates an electric field that causes oxygen ions to diffuse towards the bulk titanium, leading to the formation of TiO₂. The sequences of reactions are shown below:

Dissolution and transfer of electrons to absorbed oxygen ions

Ti═Ti⁴⁺+4e⁻

Evolution of the oxygen from the anode surface

4OH⁻═O2+2H₂O+4e⁻

Formation of the passive film on the anode surface

Ti+2OH⁻═TiₓO_{y}+H₂O+2e⁻.

### Contact Angle and Surface Energy

A Kyova contact angle meter DM CE-1 was used to determine the contact angle and surface energy of the alloys by a sessile drop method using deionized water (polar), ethylene glycol (neutral), and diiodomethane (non-polar) as probe liquids.

Young-Dupré Equation

Young equation gives the correlation between the surface free energy (SFE) of the liquid γ_{L}, surface free energy of the solid γ_{S}, interfacial free energy between solid and liquid γ_{SL}, and contact angle between the probe liquid and the examined surface θ as given by the equation below.

γ_{S}−γ_{L} cos θ+γ_{SL}.

### X-Ray Diffraction (XRD) Analysis

The microstructure and crystallinity of oxide layers on the surface of MEP treated nitinol alloys were determined using a Siemens 500D X-ray Diffractometer (XRD).

### X-Ray Photo Electron Spectroscopy (XPS) Analysis

The amount of Ti, Ni, Cr, Ta, and their respective oxides and their thickness on the surface of both MP and MEP treated nitinol alloys were determined with a PHI Quantera scanning XPS microprobe using a monochromatic Al Kα X-ray radiation.

### Platelet Adhesion Test

A parallel plate laminar flow chamber was used to investigate the adhesion of blood components on the surface of the implant materials.

Each biomaterial is placed in a recessed cavity of five flow chambers. Blood containing fluorescently labeled platelets (mepacrine) was passed over each sample for 35 minutes to measure platelet deposition.

The loop consisted of a peristaltic pump to maintain blood flow at 160 ml/min, silicon tubes to connect the flow chambers, a blood reservoir, and a water bath to maintain the temperature of blood at 37° C. Prior to hemocompatibility testing, metallic samples were ultrasonically cleaned for 5 minutes in deionized water followed by cleaning in 70% ethanol for 5 minutes to get rid of impurities and foreign particles on their surface. Once all the samples were placed in the chambers, phosphate buffer saline (PBS) solution was passed through the loop for 10 minutes. Approximately 500 ml of freshly collected whole porcine blood was mixed with 150 ml of sodium citrate anticoagulant to avoid coagulation. 333.5 ml of 10 mM mepacrine dye solution was added for every 500 ml of whole porcine blood to fluorescently label the platelets. The blood was passed over the metallic samples in the loop for 35 minutes. After each run, samples were extracted and carefully washed 3 times with PBS to remove any residual blood components. Platelets adhered on to these samples were observed under a Nikon Eclipse E 200 fluorescent microscope and quantified using Image J software.

### EXAMPLES

Following are examples that illustrate embodiments and procedures for practicing the invention. These examples should not be construed as limiting.

### Example 1

### MEP Decreases Platelet Adhesion on Ternary Nitinol Alloys

The amount of platelets adhered on MEP NiTi10Ta (33 cells/mm²) and MEP NiTi5Cr (42 cells/mm²) was lower as compared with that on mechanically polished NiTi10Ta (48 cells/mm²) and MP NiTi5Cr (53 cells/mm²). In order to establish whether the magnitude of platelet adhesion per unit surface for each alloy was significantly different, Tukey's HSD (honestly significant difference) test was conducted. It revealed that platelet adhesion on MEP nitinol alloys was significantly different (p<0.05) from that on untreated nitinol alloys.

FIG. 2 shows platelet adhesion on MP and MEP treated nitinol alloys with respect to surface chemistry (oxide), work of adhesion (W), and contact angle (CA). The lowest concentration of platelet adhesion was observed on MEP NiTi10Ta. This can be the result of a) structure of the oxide layer, b) chemistry of the oxide and/or c) the amount of oxide which influences the hemocompatibility.

Platelet adhesion appears to be dependent on the hydrophobicity of the material's surface. As shown in FIG. 2, MEP resulted in an increase in CA and a decrease in platelet adhesion. The work of adhesion (W) which is derived from CA measurement is directly proportional to platelet adhesion. MEP nitinol alloys had a lower surface free energy (SFE ˜63 mJ/m²) and W (75-78 mJ/m²) as compared with that of MP nitinol alloys of SFE (38-40 mJ/m²) and W (95-102 mJ/m²).

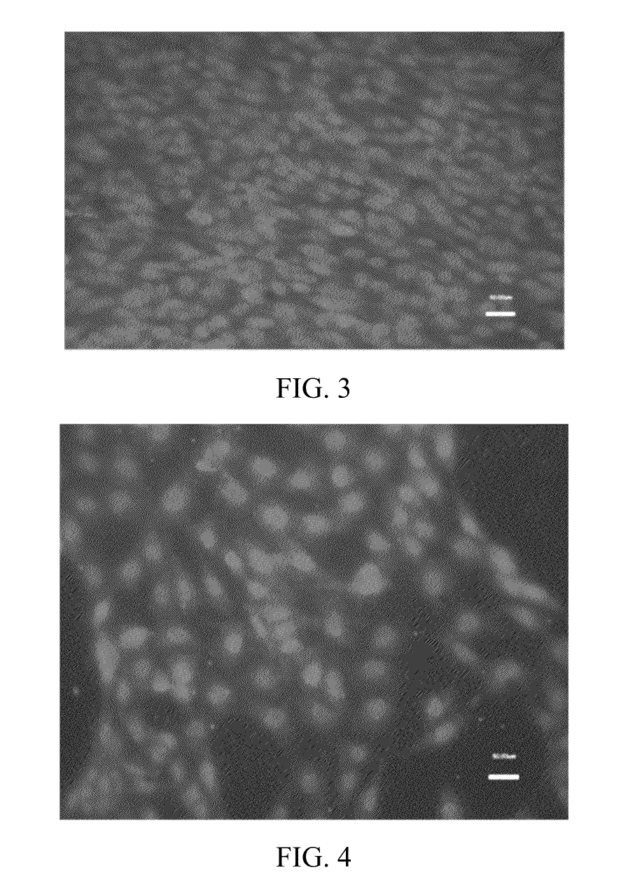

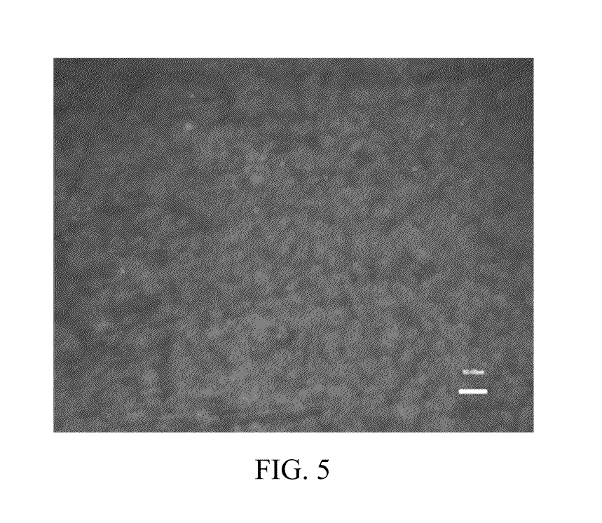

In addition to the achieving reduced platelet adhesion on MEP ternary nitinol alloys, confluent growth of Human umbilical vein endothelial cells (HUVEC) was also observed. FIGS. 3, 4, and 5 show highlighted HUVE cell nuclei and cell mitochondria.

### Example 2

### MEP Treatment Produces Specific Titanium Oxides on Ternary Nitinol Surface

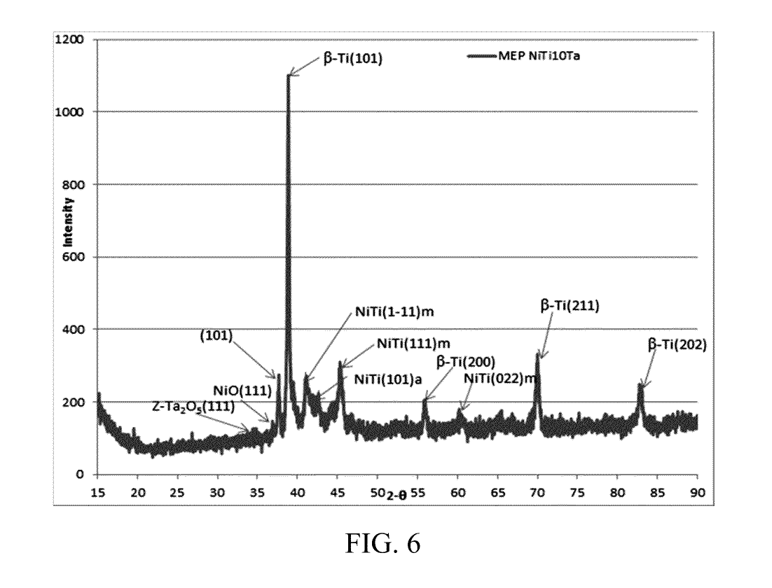

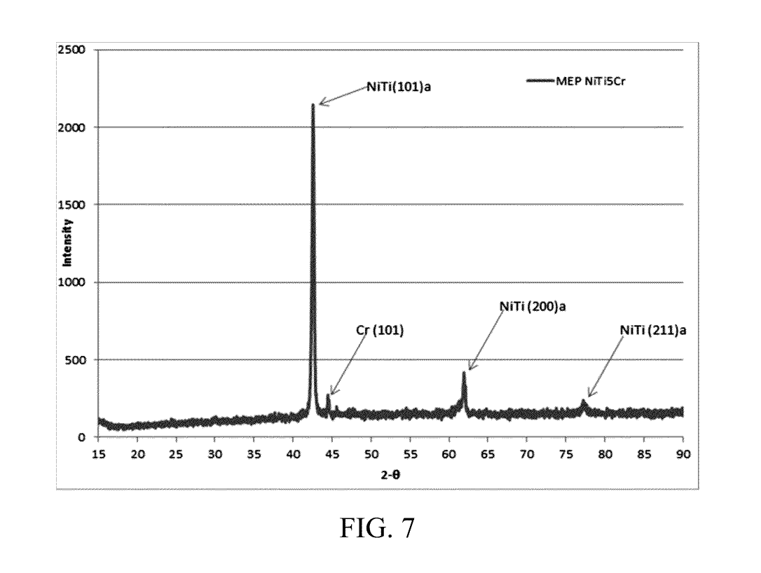

Rutile is the common titanium oxide formed on binary nitinol. The crystal structure of titanium oxide on MEP NiTi10Ta was anatase, which is amorphous, whereas that on MEP NiTi5Cr was rutile, which is more crystalline in nature. This variation in crystallography may be attributed to the relative atomic size of tantalum with respect to nickel and titanium. Furthermore, nano hardness analysis revealed that NiTi5Cr (6.2 GPa) was harder than NiTi10Ta (3 GPa). The XRD analysis as shown in FIGS. 6 and 7 confirmed the crystal structure of titanium oxide on MEP nitinol alloys.

XPS analysis of MEP nitinol alloys revealed the formation of a compact oxide layer on the surface of MEP NiTi10Ta (about 10 nm) as compared with MP NiTi (about 23 nm) and MP NiTi10Ta (about 29 nm) and a higher oxide content on MEP NiTi10Ta (about 15 at %) despite the thinner layer as compared with MP NiTi (about 11 at %) and MP NiTi10Ta (about 12 at %). Additionally, Cr₂O₃ and Ta₂O₅ were observed on MEP NiTi5Cr and MEP NiTi10Ta respectively.

Binary and ternary nitinol alloys of composition Ni51Ti49, Ni48Ti47Cr5 and Ni46Ti44Ta10 were prepared by arc melting (AM) and subjected to MEP treatment as described in the United States Patent Application Publication No. 2012/0093944, the contents of which are incorporated herein in its entirety.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. An alloy comprising titanium, nickel, and a third metal element, wherein the third metal element is tantalum or chromium.

2. The alloy of claim 1, wherein nickel and titanium together comprise 80 atomic percent to 99 atomic percent and the third metal element comprises from 1 atomic percent to 20 atomic percent.

3. An implant comprising the alloy of claim 1.

4. The implant of claim 3, wherein the alloy is present on the surface of the implant.

5. The implant of claim 3, wherein the surface of the implant is treated with magnetoelectropolishing.

6. The implant of claim 3, wherein the implant is a cardiovascular implant or an endovascular implant.

7. The implant of claim 6, wherein the implant is a vascular access device, needle, heart valves prosthesis, catheter, arteriovenous fistula, bare metal stent, drug eluting stent, blood clot retriever, vena cava filter, or endoscope.
